(19) United States
(12) Patent Application Publication
(10) Pub. No.: US 2002/0115087 A1
(43) Pub. Date: Aug. 22, 2002

(54) Method for identifying type 2 hypercarbic respiratory failure associated with community acquired pneumonia

(76) Inventors: Richard Glenn Wunderink, Germantown, TN (US); Grant William Waterer, Yokine (AU)

Correspondence Address: Licata & Tyrrell P.C., 66 E. Main Street, Marlton, NJ, 08053, US

(21) Appl. No.: 09/974,394

(22) Filed: Oct. 10, 2001

Related U.S. Application Data
(60) Provisional application No. 60/238,977, filed on Oct. 10, 2000.

(51) Int. Cl. C12Q 1/68

(52) U.S. Cl. 435/6

(57) ABSTRACT

A method of identifying patients having a predisposition to Type 2 Hypercarbic Respiratory Failure and/or an increased requirement for mechanical ventilation when they have community-acquired pneumonia (CAP) through detection of IL 10 gene polymorphisms at the −1082 site. The method can be used to diagnose predisposition or susceptibility to Type 2 Hypercarbic Respiratory Failure and/or an increased requirement for mechanical ventilation in patients with CAP. Compositions for said diagnosis are provided. Methods of treatment of CAP in such patient are provided, comprising identifying an individual having a predisposition or susceptibility to Type 2 Hypercarbic Respiratory Failure and/or an increased requirement for mechanical ventilation and subsequently treating that individual for such condition.

### INTRODUCTION

This application claims the benefit of priority from U.S. provisional application Ser. No. 60/238,977 filed Oct. 10, 2001.

### FIELD OF THE INVENTION

This invention relates methods based upon a polymorphism in the Interleukin 10 (IL-10) gene, more specifically, guanine (G) at the −1082 site rather than the usual adenine (A). More specifically, this invention relates to a method for identification of patients having a predisposition to Type 2 Hypercarbic Respiratory Failure and/or an increased requirement for mechanical ventilation with Community-Acquired Pneumonia (CAP), by screening for the presence of this polymorphism at the −1082 site. The invention also relates to compositions for screening for the polymorphism and improved treatment choices for patients identified with such susceptibility by the method of the present invention. This invention also relates to novel compound screening and treatments.

### BACKGROUND OF THE INVENTION

Pneumonia is a common clinical entity, particularly among the elderly. A thorough understanding of the epidemiology and microbiology of Community-Acquired Pneumonia (CAP) is essential for appropriate diagnosis and management. Although the microbiology of CAP has remained relatively stable over the last decade, there is new information on the incidence of atypical pathogens, particularly in patients not admitted to hospital, and new information on the incidence of pathogens in cases of CAP and in CAP in the elderly. Recent studies have provided new data on risk factors for mortality in CAP, which can assist the clinician in decisions about the need for hospital admission. The emergence of antimicrobial resistance in Streptococcus pneumonia, the organism responsible for most cases of CAP, has greatly affected the approach to therapy, especially in those patients who are treated empirically. Guidelines for the therapy of CAP have been published by the American Thoracic Society, the British Thoracic Society, and, most recently, the Infectious Diseases Society of America and others. These guidelines differ in their emphasis on empirical versus pathogenic-specific management.

CAP remains a significant health problem and patients continue to die despite receiving appropriate antibiotic therapy. Modification of the host immune response, both anti- and pro-inflammatory approaches, has yet to live up to the promise of improved outcome. Despite this, there is significant reason for optimism. Some immunomodulatory therapies clearly have efficacy in some patients. As the understanding of the immune response to pneumonia improves our ability to tailor specific therapies for individual patients will also improve, hopefully avoiding the deleterious effects that have so far prevented the development of an effective immune based therapy. The possibility of delivering cytokines directly to the lung, is a particularly promising way of achieving the desired pulmonary effect without systemic side effects. Corticosteroids are currently unique in that they have a proven role in the therapy of pneumonia due to P. carinii. The development of pathogen specific therapies, such as INF for L. pneumophila, based on an improved understanding of host-pathogen interactions, are awaited.

The past 20 years has seen an explosion in our knowledge of human immunology and we are only now beginning to explore the therapeutic possibilities this has made available. The next 10 years promises to finally provide a significant advance in the therapy of pneumonia, the first substantial gain since penicillin.

In light of the prevalence of CAP and the evolution of resistance in the most common bacterial CAP pathogen, physicians advise obtaining specimens for culture of CAP pathogens and analyzing patterns of susceptibility, especially of S. pneumonia, in their communities, using antibiotics appropriately and prudently, according to prevailing susceptibilities when empirical treatment is called for, and immunizing susceptible patients with pneumococcal and influenza vaccines. This is because the mortality of patients with CAP approaches or may exceed 20%, compared to less than 1% for patients with non-CAP (Fine et al. New Engl. J. Med. 1997.336:243-at the −1082 site, British Thoracic Society, Q. J. Med. 1987.239:192-220, Niederman et al. Am. Rev. Resp. Dis. 1993.148:1418-1426). In such cases an ability to diagnosis a patient having CAP susceptibility to Type 2 Hypercarbic respiratory failure would be of distinct advantage and may lead to improved outcomes and lower medical costs for such patients by correct intervention based on the diagnosis.

IL-10 is a cytokine largely responsible for down regulating the immune response to inflammatory stimuli (such as infection). It also plays a critical role in directing the immune response towards a TH2 (antibody producing) immune response rather than a TH1 (cytotoxic) immune response. The GG genotype of IL-10 is associated with IL-10 hypersecretion. However, it is a surprising feature of the present invention to be able to diagnose the susceptibility to type 2 Hypercarbic Respiratory Failure, and/or an increased likelihood of requiring mechanical ventilation, in a patient having CAP by the method of the present invention.

### BRIEF SUMMARY OF THE INVENTION

It is an object of the present invention to improve the clinical outcome in patients with a predisposition to type 2 Hypercarbic Respiratory Failure and/or an increased requirement for mechanical ventilation having CAP identified by the methods of this invention. It is a particular object of the invention to provide a method of identification of predisposition or susceptibility to type 2 Hypercarbic Respiratory Failure and/or an increased requirement for mechanical ventilation to aid in the effective treatment of such CAP patients. A further object is to provide, following such identification, a method of identifying patients for alternative management of such type 2 Hypercarbic Respiratory Failure and/or an increased requirement for mechanical ventilation before the disease becomes significantly established. Thus, the invention also relates to compositions for screening for the IL-10 polymorphism at the −1082 site and improved treatment choices for patients diagnosed as being susceptible to type 2 Hypercarbic Respiratory Failure and/or an increased requirement for mechanical ventilation when they have CAP by the method of the present invention.

The invention also relates to novel compound screening methods and treatments based on the association made herein. Other preferred embodiments of the present invention will be apparent to one of ordinary skill in light of the following description of the invention and of the claims.

### DETAILED DESCRIPTION OF THE INVENTION

In a first aspect, the invention provides a method of identifying a disease associated with a genetic polymorphism (guanine (G) at the at the −1082 site rather than adenine (A)) in a IL-10 gene in an animal predisposed or susceptible to said disease, said method comprising determining the genotype of IL-10 in said animal. The first aspect of the invention further provides a method of identifying an animal, including human, predisposed or susceptible to a disease associated with a genetic polymorphism in an IL-10 gene, said method comprising determining the genotype of said IL-10 gene in said animal. In an embodiment of the invention, the method comprises screening for an individual at risk of a condition or disease such as type 2 Hypercarbic Respiratory Failure and/or an increased requirement for mechanical ventilation in patients when they have CAP that is correlated with a IL-10 gene polymorphism at the −1082 site. IL-10 is well known and widely described in the literature. (See e.g., Turner D. M., Williams D. M., Sankaran D., Lazarus M., Sinnott P. J., Hutchinson IV An investigation of polymorphism in the interleukin-10 gene promoter. Eur. J. Immunogenet. 1997.24:1-8)

The invention is based upon the observation reported herein of a correlation between polymorphisms in the IL-10 gene, specifically at position −1082 site, and predisposition or susceptibility to type 2 Hypercarbic Respiratory Failure and/or an increased requirement for mechanical ventilation in patients when they have CAP. The invention is of advantage in that by screening for the presence of the polymorphism it is possible to identify individuals likely to have a genetic predisposition or susceptibility to the disease. It may also result in substantially different management, both prevention and treatment, if CAP occurs, with subsequent substantial improvement in mortality and morbidity from CAP in such susceptible patients.

In an embodiment of the invention, diagnosis is carried out by determining whether a IL-10 gene contains the G allele at the −1082 site. Possessing a fragment that contains the G at the −1082 site correlates with increased risk of predisposition or susceptibility to type 2 Hypercarbic respiratory failure and an increased likelihood of requiring mechanical ventilation in patients with CAP. As a human genome contains two IL-10 genes, one on each of a pair of chromosomes, an individual can accordingly be found to be homozygous or heterozygous for the risk polymorphism, or to lack the risk polymorphism.

Genotypic and allelic frequencies of this invention are readily determined by a number of methods known to those skilled. Examples used in the present invention are shown in the Example below and include using PCR amplification, heteroduplex generation and gel electrophoresis.

The method conveniently comprises amplifying a fragment of a IL-10 gene to produce copies and determining whether copies of the fragment contain the polymorphism.

Another suitable technique is to amplify the fragment using PCR techniques, producing copies of a fragment that is at least 500 base pairs in length, preferably at least 600 base pairs in length. It is preferred that the PCR primers are selected so as to amplify a region of the gene that is about 740 base pairs in length. PCR techniques are well known in the art and it would be within the ambit of a person of ordinary skill in this art to identify primers for amplifying a suitable section of the applicable exon of the IL-10 gene. PCR techniques are described for example in EP-A-0200362 and EP-A-0201 184. In a further embodiment of the invention, the diagnostic method comprises analysis of the IL-10 gene using single strand conformational polymorphism (SSCP) mapping to determine whether the IL-10 gene is the risk or the non-risk form.

As described above, in preferred embodiments of the first aspect of the invention, the method comprises screening a IL-10 gene, and this screening is conveniently carried out by any one of a number of suitable techniques that are known in the art, and may be conveniently selected from amplification of a nucleic acid sequence located within the IL-10 gene, Southern blotting of regions of the gene and single strand conformational polymorphism mapping of regions within the gene or as described in the example below. The genotype in that region is also optionally determined using a variety of methods including hybridization probes adapted selectively to hybridize with the particular polymorphism of the IL-10 gene at the −1082 location which is associated with predisposition or susceptibility to disease. A probe used for hybridization detection methods must be in some way labeled so as to enable detection of successfully hybridization events. This is optionally achieved by in vitro methods such as nick-translation, replacing nucleotides in the probe by radioactively labeled nucleotides, or by random primer extension, in which non-labeled molecules act as a template for the synthesis of labeled copies. Other standard method of labeling probes so as to detect hybridization are known to those skilled in this art.

According to a second aspect of the invention there is provided a method of diagnosis and therapy comprising identifying CAP by methods known to those of skill in the art and identifying predisposition or susceptibility to type 2 Hypercarbic Respiratory Failure and/or an increased requirement for mechanical ventilation according to the method of the invention and treating an individual having such susceptibility thereby treating to prevent or lessen CAP.

Known therapies for CAP can be effective in halting advancement of the disease, or at least slowing the advancement. IL-10 −1082 gene analysis may also lead to more appropriate placement of patients into intensive care/critical care units, mechanical ventilation, an important factor in optimizing survival from CAP in patients diagnosed by the method of this inventions as susceptible to type 2 Hypercarbic Respiratory Failure and/or an increased requirement for mechanical ventilation. It is thus an advantage of the invention that early diagnosis of susceptibility to type 2 Hypercarbic Respiratory Failure and an increased requirement for mechanical ventilation in patients when they have CAP is improved, so that preventative therapy can be started as soon as possible, optimizing any interventions potential (such as mechanical ventilation, vaccination and/or immunomodulatory therapy) for affecting outcome. The decision of a physician on how and whether to initiate therapy in anticipation of the disease can be taken with increased confidence.

A variety of suitable treatments of CAP and associated type 2 Hypercarbic Respiratory Failure and/or an increased requirement for mechanical ventilation are described in the art and herein. See also, Hirani and MacFarlane Thorax 1997.52:17-21, Pachon J. et al. Am. Rev. Resp. Dis. 1990.142:369-373, Ruiz M. et al. Am. J. Respir. Crit. Care. Med. 1999.160:923-929, Leeper and Torres Clin. Chest. Med. 1995.16:155-171. Other treatments will be known to persons of skill in the art.

A third aspect of the invention provides a composition for use in identifying a disease associated with a genetic polymorphism in a IL-10 gene in an animal predisposed or susceptible to said disease, said composition comprising one or more primer nucleic acid molecules adapted to amplify a portion of a IL-10 gene selected from a portion of the gene around the at the −1082 site location.

A third aspect of the invention also provides a composition for use in identifying an animal predisposed or susceptible to a disease associated with a genetic polymorphism in a IL-10 gene, said compositions comprising one or more primer nucleic acid molecules adapted to amplify a portion of the IL-10 gene selected from a portion of the gene around the at the −1082 site location.

The composition of the third aspect of the invention may comprise a nucleic acid molecule capable of identifying the −1082 risk polymorphism in said IL-10 gene, said polymorphism being indicative of a risk genotype in said animal.

A further embodiment of the third aspect of the invention provides a composition for identification of predisposition or susceptibility to type 2 Hypercarbic Respiratory Failure and/or an increased requirement for mechanical ventilation in patients when they have CAP, comprising means for determining genotype of a IL-10 gene of an individual, for example whether an individual is homozygous or heterozygous for polymorphic variants of a IL-10 gene at the −1082 location such as the method provided in the example herein (G allele).

In an embodiment of the invention, a composition comprises PCR primers adapted to amplify a DNA sequence within and around the IL-10 gene at the −1082 site polymorph location, wherein alternative versions of the gene are distinguished one from another.

In a further aspect of the invention there is provided a diagnostic kit comprising a composition such as described above and an indicator composition for indicating how possessing a polymorphic version of a IL-10 gene (G allele) correlates with the predisposition or susceptibility to type 2 Hypercarbic Respiratory Failure and/or an increased requirement for mechanical ventilation in patients with CAP.

Diagnostic kits are typically accompanied by or comprise a chart or other visual aid for assistance in interpreting the results obtained using the kit. Suitable indicator compositions for use in the kit of the invention include a leaflet or other visual reminder that possessing the risk polymorphism version of a IL-10 gene (G allele) correlates with the predisposition or susceptibility to susceptible to type 2 Hypercarbic Respiratory Failure and/or an increased requirement for mechanical ventilation in patients with CAP.

In a still further aspect of the invention there is provided use, in the manufacture of means for identifying whether an individual has a predisposition or susceptibility to type 2 Hypercarbic Respiratory Failure and/or an increased requirement for mechanical ventilation in patients with CAP, of PCR primers adapted to amplify a region around at the −1082 site in the IL-10 gene. Alternative versions of the gene are typically distinguished one from another by means known to those skilled in the art.

Multiple techniques exists and are known to one skilled in the art in the manufacture of means for identifying whether an individual has a predisposition or susceptibility to type 2 Hypercarbic Respiratory Failure and/or an increased requirement for mechanical ventilation in patients with CAP by determining the risk polymorphism of this invention, by using, for example, PCR primers adapted to amplify a region around at the −1082 site in the IL-10 gene. One can use heteroduplex formation where different lengths of DNA are generated depending on whether a G or A is present at the −1082 site, and the IL-10 −1082 genotype can be subsequently determined by gel electrophoresis.

According to the invention, an individual who is homozygous for a risk polymorphism, that is to say homozygous for a version of at the −1082 site (GG), is classified as being at highest risk. An individual being heterozygous (GA) is classified as having moderate risk.

Optionally, the assessment of an individual's risk factor according to any aspect of the invention is calculated by determining the genotype of a IL-10 gene polymorphism and combining the result with analysis of other known genetic or physiological or dietary or other risk indications. The invention in this way provides further information on which measurement of an individual's risk can be based.

In another embodiment of the invention, the results of the genotyping done herein are used, along with other diagnostics measures and disease parameters, by treatment providers to determine the best course of treatment for the CAP patient having been identified as susceptible to type 2 Hypercarbic Respiratory Failure, and/or an increased requirement for mechanical ventilation by the methods of this invention.

The IL-10 polypeptide described in the present invention (G allele at the −1082 site) may be beneficially employed in a screening process for compounds which stimulate (agonists) or inhibit (antagonists, or otherwise called inhibitors) the synthesis or action of the IL-10 polypeptide. The IL-10 polypeptide may also be employed in a screening process for compounds which mimic the agonist or antagonist properties of the IL-10 polypeptide. Thus, the polypeptide encoded by IL-10 (G allele at the −1082 site) may also be used to assess and identify agonist or antagonists from, for example, cells, cell-free preparations, chemical libraries, and natural product mixtures. These agonists or antagonists may be natural substrates, ligands, receptors, etc., as the case may be, of the polypeptide of the present invention; or may be structural or functional mimetics of the polypeptide of the present invention. See Coligan et al., Current Protocols in Immunology 1(2):Chapter 5 (1991).

IL-10 proteins are ubiquitous in the mammalian host and are responsible for many biological functions, including many pathologies. Accordingly, it is desirous to find compounds and drugs which stimulate IL-10 polypeptide (G allele at −1082) on the one hand and which can inhibit the function of IL 10 polypeptide (G allele at −1082) on the other hand.

In general, such screening procedures may involve identifying, generating and using appropriate cells which express the receptor of the IL-10 polypeptide on the surface thereof. Such cells include cells from mammals, yeast, Drosophila or E. coli. Such cells may be identified, for example, by direct binding methods using radiolabeled or fluorescently tagged IL-10 polypeptide (G allele at −1082). Cells expressing the IL-10 polypeptide receptor (or cell membrane containing the expressed polypeptide) are then contacted with a test compound to observe binding, or stimulation or inhibition of a functional response. Alternatively, the cDNA for the IL-10 polypeptide receptor may be cloned by the above direct binding methods using expression cloning or purification methods known in the art, and its extracellular domain expressed as a secreted or membrane protein. The soluble or membrane bound receptor can then be used to identify agonists or antagonists via direct binding methods.

The assays may simply test binding of a candidate compound wherein adherence to the cells bearing the IL-10 polypeptide receptor is detected by means of a label directly or indirectly associated with the candidate compound or in an assay involving competition with a labeled IL-10 polypeptide. Further, these assays may test whether the candidate compound results in a signal similar to that generated by binding of the IL-10 polypeptide, using detection systems appropriate to the cells bearing the IL-10 polypeptide receptor at their surfaces. Inhibitors of activation are generally assayed in the presence of a known agonist and the effect on activation by the agonist by the presence of the candidate compound is observed. Standard methods for conducting such screening assays are well understood in the art.

Examples of potential IL-10 polypeptide antagonists include antibodies or, in some cases, oligonucleotides or proteins which are closely related to the ligands, substrates, receptors, etc., as the case may be, of the IL-10 polypeptide, e.g., a fragment of the ligands, substrates, receptors, or small molecules which bind to the target receptor of the present invention but do not elicit a response, so that the activity of the polypeptide is prevented. Preferred are those that can access and effect cellular function.

This invention provides methods of treating an abnormal conditions related to both an excess of and insufficient amounts of IL-10 polypeptide (G allele at −1082 site) activity.

If the activity of IL-10 polypeptide is in excess as is believed to be the case in the present invention, several approaches are available. One approach comprises administering to a subject an inhibitor compound (antagonist) as herein above described along with a pharmaceutically acceptable carrier in an amount effective to inhibit activation by blocking binding of the IL-10 polypeptide to its target receptor, or by inhibiting a second signal, and thereby alleviating the abnormal condition, i.e., susceptibility to type 2 Hypercarbic Respiratory Failure, and/or an increased requirement for mechanical ventilation in patients with CAP.

In another approach, soluble forms of IL-10 polypeptides (G allele at −1082) capable of binding its receptor in competition with endogenous IL-10 polypeptide may be administered. Typical embodiments of such competitors comprise fragments of the IL-10 polypeptide.

In still another approach, expression of the gene encoding endogenous IL-10 polypeptide can be inhibited using expression blocking techniques. Known such techniques involve the use of antisense sequences, either internally generated or separately administered. See, for example, O'Connor, J. Neurochem. 1991.56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al. Nucleic Acids Res. 1979.6:3073; Cooney et al. Science 1988.241:456; Dervan et al. Science 1991.251:1360. These oligomers can be administered per se or the relevant oligomers can be expressed in vivo.

For treating abnormal conditions related to an under-expression of IL-10 and its activity, several approaches are also available. One approach comprises administering to a subject a therapeutically effective amount of the IL-10 polypeptide or a compound, i.e., an agonist or mimetic as described above, in combination with a pharmaceutically acceptable carrier, to thereby alleviate the abnormal condition. Alternatively, gene therapy may be employed to effect the endogenous production of IL-10 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T. Strachan and A. P. Read, BIOS Scientific Publishers Ltd. (1996).

All such agonists and antagonists are administered in amounts effective to treat the condition and in pharmaceutically acceptable carriers. Techniques for determining effective amounts and carriers are well known to those of skill in the art.

It will be readily apparent to one of ordinary skill in the relevant art that other suitable modifications and adaptations to the methods and applications described herein are obvious and may be made without departing from the scope of the invention or any embodiment thereof. Having now described the present invention in detail, the same will be more clearly understood by reference to the following example, which is included herewith for purposes of illustration only and is not intended to be limiting of the invention.

### EXAMPLE 1

Methods: A prospective cohort study of CAP. Type 2 Hypercarbic respiratory failure (T2RF) was defined as an 02 saturation<90% (room air) with a pC02>44 mmHg. Septic shock (SS) was defined using ACCP-SCCM criteria. Genotype analysis was performed using heteroduplex formation and subsequent non-deforming polyacrylamide gel electrophoresis. Genotype frequencies are given in the order of AA/GA/GG.

Results: 188 CAP patients were genotyped. Overall genotype frequency was 0.36/0.46/0.18. The proportion developing SS in each genotype was 0.13/0.07/0.15 (p=NS). GG homozygotes had a greater proportion of T2RF (0.17 vs 0.06, p=0.04) and a greater need for mechanical ventilation (0.32 vs 0.13, p=0.01) than non GG homozygotes. Length of hospital stay in GG homozygotes was 10.3 days vs 7.5 days (p=0.06).

Conclusion: GG gentoype (and GA) at the IL-10 −1082 polymorphism site is associated with a greater likelihood of T2RF and a greater need for mechanical ventilation.

What is claimed is:

1. A method of identifying a predisposition or susceptibility to Type 2 Hypercarbic Respiratory Failure and/or an increased requirement for mechanical ventilation in an animal when it has community-acquired pneumonia (CAP) associated with a genetic polymorphism in a IL-10 gene at the −1082 locus, said method comprising determining the genotype of said IL-10 gene in said animal, and identifying predisposition or susceptibility to Type 2 Hypercarbic Respiratory Failure and/or an increased requirement for mechanical ventilation in an animal when it has CAP based on said genotype.

2. The method of claim 1 comprising determining whether an individual is homozygous or heterozygous for alternative versions of a IL-10 gene polymorphism at the −1082 locus.

3. A method of identifying a predisposition or susceptibility to Type 2 Hypercarbic Respiratory Failure and/or an increased requirement for mechanical ventilation in patients with community-acquired pneumonia (CAP), the method comprising determining whether the individual possesses a polymorphic risk version of a IL-10 gene at the −1082 locus, a polymorphic risk version of the gene being one that has an G at site at the −1082 site, the method comprising:
(a) using heteroduplex formation and subsequent non-deforming polyacrylamide gel electrophoresis of the IL-10 gene thereby determining if a G is present at the −1082 site and not A,
(b) testing whether the copies contain an A or a G at site at the −1082 site through known differences in how such fragments migrate on a gel, and thereby determining whether the individual is homozygous or heterozygous for a polymorphic risk version of the gene, and
(c) identifying the presence of susceptibility to Type 2 Hypercarbic Respiratory Failure and/or an increased requirement for mechanical ventilation in patients with CAP as greatest if that individual is homozygous for the polymorphic risk version of the gene at the −1082 site (GG), moderate if that individual is heterozygous for the polymorphic risk version at the −1082 site (GA), and least if that individual lacks the polymorphic risk version at the −1082 site (AA).

4. A method of managing and treating patients with a predisposition to or who are susceptible to Type 2 Hypercarbic Respiratory Failure and/or an increased requirement for mechanical ventilation when they have CAP, comprising determining whether the individual possesses a polymorphic risk version of a IL-10 gene at the −1082 locus, a polymorphic risk version of the gene being one that has a G at site at the −1082 site, wherein the management and treatment of such patient having such polymorphism are promptly treated and managed as patients having a predisposition to Type 2 Hypercarbic Respiratory Failure and/or an increased requirement for mechanical ventilation when they have community-acquired pneumonia (CAP).

5. A method of screening to identify compounds which stimulate or inhibit the synthesis or action of the IL-10 polypeptide having a G allele at the −1082 site comprising screening compounds in an IL-10 polypeptide having the G allele at the −1082 site, assay and identifying those compounds which stimulate IL-10 as agonists and those compounds which inhibit such IL-10 as antagonists.

6. A agonist or antagonist identified by the method of claim 5.

7. A method of treating patients comprising identifying a patient with a predisposition to or who are susceptible to Type 2 Hypercarbic Respiratory Failure and/or an increased requirement for mechanical ventilation when they have CAP by identifying the G allele in a IL-10 gene at the −1082 locus in such patient, and administering such patient an effective amount of an antagonist identified in claim 5 in a pharmaceutically acceptable carrier.
